(19) United States
(12) Patent Application Publication
(10) Pub. No.: US 2010/0234366 A1
(43) Pub. Date: Sep. 16, 2010

(54) Use of Strobilurins for the Treatment of Disorders of Iron Metabolism

(75) Inventors: Bennard van Ravenzwaay, Altrip (DE); Werner Mellert, Hassloch (DE); Georgia Coelho Palermo Cunha, Limburgerhof (DE); Klaus Deckardt, Heddesheim (DE); Heinz Kieczka, Grunstadt (DE)

Correspondence Address: BRINKS, HOFER, GILSON & LIONE, P.O. BOX 1340, MORRISVILLE, NC, 27560, US

(73) Assignee: BSF SE, Ludwigshafen (DE)

(21) Appl. No.: 12/294,480

(22) PCT Filed: Mar. 27, 2007
(86) PCT No.: PCT/EP2007/052917

§ 371 Date: Sep. 25, 2008

(30) Foreign Application Priority Data
Mar. 29, 2006 (EP) 06111933.5

(51) Int. Cl. A61K 31/539; A61K 31/24; A61P 39/02; A61P 3/00; A61K 31/165; A61K 31/44; A61K 31/415; A61K 31/505

(52) U.S. Cl. 514/229.2; 514/539; 514/619; 514/538; 514/345; 514/406; 514/269

(57) ABSTRACT

Strobilurin derivatives can be employed for the treatment and/or prevention of disorders of iron metabolism in mammals.

The present invention relates to the treatment and prevention of disorders of iron metabolism by use of strobilurins and their synthetic analogs.

Iron is an element which is widespread in nature and which can be detected in virtually all cells of the animal and plant organism. As essential trace element, iron and its compounds play an important part in healthy nutrition of mammals, including humans. Various iron-deficiency disorders occur naturally in humans and animals, as well as in plants, but disorders of iron metabolism based on an elevated iron level are also observed. In the veterinary medical sector and in human nutrition it is possible to eliminate iron-deficiency disorders by intake of iron products which ordinarily comprise iron in the form of iron(II) or iron(III) compounds.

It is known that iron(II) is absorbed in humans and animals mainly in the duodenum. Iron(III) is ordinarily reduced first and only then absorbed. About two thirds of the iron in the human body is bound to hemoglobin, while one third of the iron is stored in the form of other proteins, for example in myoglobin or ferritin. Whereas most of the iron requirement in humans and animals can be met simply by reuse of iron released on degradation of hemoglobin, the remaining hemoglobin must be supplied via the diet.

It has been known for decades that iron compounds not only have a corrosive effect but, in higher dosage, may also have mucosa-irritating and acute toxic effects. Serious manifestations of toxicity may occur when the binding capacity of the storage proteins in the blood is exceeded. The underlying cell damage in the gastrointestinal tract, but also in the liver, can be explained inter alia by the formation of free radicals from water and oxygen by the transition metal ions iron(II) and iron(III). There is an increased occurrence of hydroxyl free radicals, which are strong oxidizing agents. The hydroxyl free radicals react with various organic molecules in the cell and lead to serious damage to or destruction of whole organs. The dose for acute toxicity (LD 50) for example for iron(II) sulfate is about 700 mg/kg of body weight in mice, about 300 mg/kg of body weight in rats and about 600 mg/kg of body weight in rabbits.

However, humans and animals not only experience acute poisonings, which ordinarily become manifest only some hours after the administration of iron (e.g. accidental poisonings if small children have swallowed too many vitamin tablets), chronic poisonings are also observed owing to a regular excessive supply of metal, in particular iron, in the diet (see, for example, hemosiderosis).

The typical symptoms of poisoning relate in particular to the gastrointestinal tract (for example intestinal bleeding and diarrhea), the urinary tract (discoloration of the urine), the cardiovascular system (for example metabolic acidosis and circulatory shock) and the skin (mucosal lesions, edemas).

Besides the described disorders of iron metabolism due to increased intake of iron, genetic factors also play an essential part. For example, in hemochromatosis there is found to be an increased absorption of iron in the body and deposition of iron in various organs. In such cases there is a cirrhotic transformation of pancreas and liver, often symptomatically detectable by an altered skin pigmentation and myocardial damage.

Whereas it was possible many centuries ago to help patients with disorders of iron metabolism or iron poisoning therapeutically by “phlebotomy”, different treatment methods have been developed latterly.

The therapy of metal poisoning aims firstly and chiefly at symptomatic treatment of the frequently occurring circulatory and respiratory problems, but it is secondly possible to reduce the iron levels by administering a suitable antidote. An example of an antidote employed in clinical practice is the chelating agent deferoxamine mesilate, which can be given intravenously in a dose of up to 80 mg/kg per day, although it is necessary for a low infusion rate to be maintained precisely.

One object of the present invention was to provide more effective methods of treatment which can be implemented more easily and have few side effects for disorders of iron metabolism.

It has surprisingly been found that strobilurins and their synthetic analogs can be employed effectively for the treatment and prevention of disorders of iron metabolism.

The strobilurins can be used for the preparation of a medicament for the treatment and/or the prevention of disorders of iron metabolism in mammals, particularly in humans.

Strobilurins have been known for decades as natural products. They are produced in nature by fungi of the genus Strobilurus, but can also be prepared satisfactorily by a synthetic route. Since the naturally occurring strobulin A is easily decomposed on exposure to light, in recent years numerous derivatives have been prepared synthetically, and some of them have been used commercially as active ingredients for fungicidal preparations.

Strobilurins have to date been employed for preventive control of fungal pests in various cereal species. They act in the mitochondria and interfere with the process of cellular respiration by leading to a stoppage of electron transport in the respiratory chain.

EP-A 477631, WO 97/15552 and WO 03/075663 describe for example suitable strobilurin derivatives.

The present invention thus relates to the use of strobilurins as medicaments in general and in particular for the treatment and/or prevention of disorders of iron metabolism in mammals, especially in humans.

The invention also relates to the use of strobilurine derivatives for the preparation of a medicament or pharmaceutical composition for the treatment and for prophylaxis (prevention) of disorders of irons metabolism.

Although strobilurin derivatives of diverse structure can be used, a strobilurin derivative of the general formula (I) is preferably employed according to the invention.

The compounds involved have the formula (I)

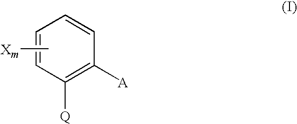

in which the substituents have the following meanings:
X halogen, C₁-C₄-alkyl or trifluoromethyl;
m 0 or 1;
   Q C(═CH—CH₃)—COOCH₃, C(═CH—OCH₃)—COOCH₃, C(═N—OCH₃)—CONHCH₃, C(═N—OCH₃)—COOCH₃, N(—OCH₃)—COOCH₃ or

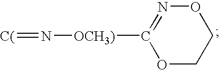

A —O—B, —CH₂O—B, —CH₂S—B, —OCH₂—B, —CH═CH—B, —C≡C—B, —CH₂O—N═C(R¹)—B, —CH₂O—N═C(R¹)—C(R²)═N—OR³,

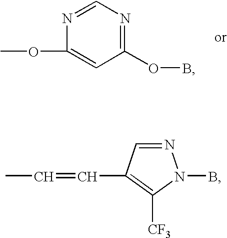

where
B phenyl, naphthyl, 5-membered or 6-membered hetaryl or 5- or 6-membered heterocyclyl, comprising one to three N atoms and/or one O or S atom or one or two O and/or S atoms, where the ring systems are unsubstituted or substituted by one to three radicals R^{a}:
   R^{a} cyano, nitro, amino, aminocarbonyl, aminothiocarbonyl, halogen, C₁-C₆-alkyl, C₁-C₆-haloalkyl, C₁-C₆-alkylcarbonyl, C₁-C₆-alkylsulfonyl, C₆-alkylsulfinyl, C₃-C₆-cycloalkyl, C₁-C₆-alkoxy, C₁-C₆-haloalkoxy, C₁-C₆-alkyloxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, phenyl, phenoxy, benzyl, benzyloxy, 5- or 6-membered heterocyclyl, 5- or 6-membered heterocyclyloxy, 5- or 6-membered hetaryl, 5- or 6-membered hetaryloxy, C(═NOR^{α})—OR^{β}, C(═NOR')—R^{α}, or OC(R^{α})₂—C(R^{β})═NOR^{β},
      where the cyclic radicals in turn are unsubstituted or substituted by one to three radicals R^{b}:
   R^{b} cyano, nitro, halogen, amino, aminocarbonyl, aminothiocarbonyl, C₁-C₆-alkyl, C₁-C₆-haloalkyl, C₁-C₆-alkylsulfonyl, C₁-C₆-alkylsulfinyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl, C₃-C₆-cycloalkenyl, phenyl, phenoxy, phenylthio, benzyl, benzyloxy, 5- or 6-membered hetaryl, 5- or 6-membered hetaryloxy or C(═NOR^{α})—OR^{β};
   R^{α}, R^{β} hydrogen or C₁-C₆-alkyl;
   R¹ hydrogen, cyano, C₁-C₄-alkyl, C₁-C₄-haloalkyl, C₃-C₆-cycloalkyl, C₁-C₄-alkoxy;
   R² phenyl, phenylcarbonyl, phenylsulfonyl, 5- or 6-membered hetaryl, 5- or 6-membered hetarylcarbonyl or 5- or 6-membered hetarylsulfonyl, where the ring systems are unsubstituted or substituted by one to three radicals R^{a},
      C₁-C₁₀-alkyl, C₃-C₆-cycloalkyl, C₂-C₁₀-alkenyl, C₂-C₁₀-alkynyl, C₁-C₁₀-alkylcarbonyl, C₂-C₁₀-alkenylcarbonyl, C₃-C₁₀-alkynylcarbonyl, C₁-C₁₀-alkylsulfonyl, or C(R^{α})═NOR^{β}, where the hydrocarbon radicals of these groups are unsubstituted or substituted by one to three radicals R^{c}:
   R^{c} cyano, nitro, amino, aminocarbonyl, aminothiocarbonyl, halogen, C₁-C₆-alkyl, C₁-C₆-haloalkyl, C₁-C₆-alkylsulfonyl, C₁-C₆-alkylsulfinyl, C₁-C₆-alkoxy, C₁-C₆-haloalkoxy, C₁-C₆-alkoxycarbonyl, C₁-C₆-alkylthio, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl, C₃-C₆-cycloalkyloxy, 5- or 6-membered heterocyclyl, 5- or 6-membered heterocyclyloxy, benzyl, benzyloxy, phenyl, phenoxy, phenylthio, 5- or 6-membered hetaryl, 5- or 6-membered hetaryloxy and hetarylthio, where the cyclic groups in turn may be partly or completely halogenated or may carry one to three radicals R^{a}; and
   R³ hydrogen,
      C₁-C₆-alkyl, C₂-C₆-alkenyl, C₂-C₆-alkynyl, where the hydrocarbon radicals of these groups are unsubstituted or substituted by one to three radicals R^{c}.

In a further embodiment, the substituents have the following meanings:

X halogen, C₁-C₄-alkyl or trifluoromethyl;
m 0 or 1; preferably 0;
Q C(═CH—CH₃)—COOCH₃, C(═CH—OCH₃)—COOCH₃, C(═N—OCH₃)—CONHCH₃, C(═N—OCH₃)—COOCH₃ or N(—OCH₃)—COOCH₃;
A —O—B, —CH₂O—B, —CH₂S—B, —OCH₂—B, —CH═CH—B, —C≡C—B, —CH₂O—N═C(R¹)—B or —CH₂O—N═C(R¹)—C(R²)═N—OR³, preferably —O—B, —CH₂OB, or CH₂O—N═C(R¹)—C(R²)═N—OR³, where
B is phenyl, naphthyl, 5-membered or 6-membered hetaryl or 5- or 6-membered heterocyclyl, comprising one to three N atoms and/or one O or S atom or one or two O and/or S atoms, where the ring systems are unsubstituted or substituted by one to three radicals R^{a}:
R^{a} is cyano, nitro, amino, aminocarbonyl, aminothiocarbonyl, halogen, C₁-C₆-alkyl, C₁-C₆-haloalkyl, C₁-C₆-alkylcarbonyl, C₁-C₆-alkylsulfonyl, C₃-C₆-cycloalkyl, C₁-C₆-alkoxy, C₁-C₆-haloalkoxy, C₁-C₆-alkyloxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, alkylaminothiocarbonyl, di-C₁-C₆-alkylaminothiocarbonyl, C₂-C₆-alkenyl, C₂-C₆-alkenyloxy, phenyl, phenoxy, benzyl, benzyloxy, 5- or 6-membered heterocyclyl, 5- or 6-membered heterocyclyloxy, 5- or 6-membered hetaryl, 5- or 6-membered hetaryloxy, C(═NOR^{α})—OR^{β} or OC(R^{α})₂—C(R^{β})═NOR^{β},
   where the cyclic radicals in turn are unsubstituted or substituted by one to three radicals R^{b}:
R^{b} is cyano, nitro, halogen, amino, aminocarbonyl, aminothiocarbonyl, C₁-C₆-alkyl, C₁-C₆-haloalkyl, C₁-C₆-alkylsulfonyl, C₁-C₆-alkylsulfinyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl, C₃-C₆-cycloalkenyl, phenyl, phenoxy, phenylthio, benzyl, benzyloxy, 5- or 6-membered hetaryl, 5- or 6-membered hetaryloxy or C(═NOR^{α})—OR^{β};
R^{α}, R^{β} are hydrogen or C₁-C₆-alkyl; in particular hydrogen or methyl;
R¹ is hydrogen, cyano, C₁-C₄-haloalkyl, C₃-C₆-cycloalkyl, C₁-C₄-alkoxy;
R² is phenyl, phenylcarbonyl, phenylsulfonyl, 5- or 6-membered hetaryl, 5- or 6-membered hetarylcarbonyl or 5- or 6-membered hetarylsulfonyl, where the ring systems are unsubstituted or substituted by one to three radicals R^{a},
   C₁-C₁₀-alkyl, C₃-C₆-cycloalkyl, C₂-C₁₀-alkenyl, C₂-C₁₀-alkynyl, C₁-C₁₀-alkylcarbonyl, C₂-C₁₀-alkenylcarbonyl, C₃-C₁₀-alkynylcarbonyl, C₁-C₁₀-alkylsulfonyl, or C(R^{α})═NOR^{β}, where the hydrocarbon radicals of these groups are unsubstituted or substituted by one to three radicals R^{c}:
R^{c} is cyano, nitro, amino, aminocarbonyl, aminothiocarbonyl, halogen, C₁-C₆-alkyl, C₁-C₆-haloalkyl, C₁-C₆-alkylsulfonyl, C₁-C₆-alkylsulfinyl, C₁-C₆-alkoxy, C₁-C₆-haloalkoxy, C₁-C₆-alkoxycarbonyl, C₁-C₆-alkylthio, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl, C₃-C₆-cycloalkyloxy, 5- or 6-membered heterocyclyl, 5- or 6-membered heterocyclyloxy, benzyl, benzyloxy, phenyl, phenoxy, phenylthio, 5- or 6-membered hetaryl, 5- or 6-membered hetaryloxy and hetarylthio, where the cyclic groups in turn may be partly or completely halogenated or may carry one to three radicals R^{a}; and
R³ is hydrogen,
   C₁-C₆-alkyl, C₂-C₆-alkenyl, C₂-C₆-alkynyl, where the hydrocarbon radicals of these groups are unsubstituted or substituted by one to three radicals R^{c}.

Active ingredients preferred for the method of the invention are those of the formula (I) in which Q is C(═CH—OCH₃)—COOCH₃, C(═N—OCH₃)—COOCH₃, C(═N—OCH₃)—CO—NH—CH₃ or N(—OCH₃)—COOCH₃, in particular C(═N—OCH₃)—CO—NH—CH₃.

Preferred meanings for B in formula (I) are phenyl, pyridyl, pyrimidinyl, triazolyl and pyrazolyl, in particular phenyl or pyridyl.

Active ingredients particularly preferred for the method of the invention are in particular those of the formulae (II) to (V) in which

V is OCH₃ and NHCH₃, in particular NHCH₃
Y is CH and N, in particular N.

Preferred active ingredients of the formula (I) in which Q is N(—OCH₃)—COOCH₃ are the compounds described in the specifications WO-A 93/15046 and WO-A 96/01256.

Preferred active ingredients of the formula (I) in which Q is C(═CH—OCH₃)—COOCH₃ are the compounds described in the specifications EP-A 178 826 and EP-A 278 595.

Preferred active ingredients of the formula (I) in which Q is C(═N—OCH₃)—COOCH₃ are the compounds described in the specifications EP-A 253 213 and EP-A 254 426.

Preferred active ingredients of the formula (I) in which Q is C(═N—OCH₃)—CONHCH₃ are the compounds described in the specifications EP-A 398 692, EP-A 477 631 and EP-A 28 540.

Preferred active ingredients of the formula (I) in which Q is C(═CH—CH₃)—COOCH₃ are the compounds described in the specifications EP-A 280 185 and EP-A 350 691.

Preferred active ingredients of the formula (I) in which A is —CH₂O—N═C(R¹)—B are the compounds described in the specifications EP-A 460 575 and EP-A 463 488.

Preferred active ingredients of the formula (I) in which A is —O—B are the compounds described in the specifications EP-A 382 375 and EP-A 398 692.

Preferred active ingredients of the formula (I) in which A is —CH₂O—N═C(R¹)—C(R²)═N—OR³ are the compounds described in the specifications WO-A 95/18789, WO-A 95/21153, WO-A 95/21154, WO-A 97/05103 and WO-A 97/06133.

Further preferred active ingredients are those of the formula (II)

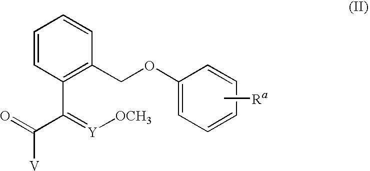

in which V is OCH₃ or NHCH₃ and Y is N, and R^{a} is halogen, C₁-C₄-alkyl, C₁-C₄-haloalkyl or C₁-C₄-haloalkoxy.

Particularly preferred active ingredients are those of the formula (II) in which V is OCH₃ or NH—CH₃ and R^{a} is halogen, methyl, dimethyl or trifluoromethyl, in particular methyl or dimethyl.

With a view to their use, the compounds listed in the following tables are particularly preferred.

TABLE I

| | |
|---|---|
| | (II) |
| 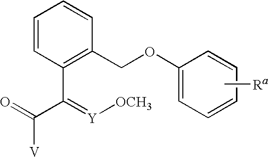 | |

| No. | V | Y | R^{a} | Literature |
|---|---|---|---|---|
| I-1 | OCH₃ | N | 2-CH₃ | EP-A 253 213 |
| I-2 | OCH₃ | N | 2,5-(CH₃)₂ | EP-A 253 213 |
| I-3 | NHCH₃ | N | 2,5-(CH₃)₂ | EP-A 477 631 |
| I-4 | NHCH₃ | N | 2-Cl | EP-A 477 631 |
| I-5 | NHCH₃ | N | 2-CH₃ | EP-A 477 631 |
| I-6 | NHCH₃ | N | 2-CH₃, 4-OCF₃ | EP-A 628 540 |
| I-7 | NHCH₃ | N | 2-Cl, 4-OCF₃ | EP-A 628 540 |
| I-8 | NHCH₃ | N | 2-CH₃, 4-OCH(CH₃)—C(CH₃)═NOCH₃ | EP-A 11 18 609 |
| I-9 | NHCH₃ | N | 2-Cl, 4-OCH(CH₃)—C(CH₃)═NOCH₃ | EP-A 11 18 609 |
| I-10 | NHCH₃ | N | 2-CH₃, 4-OCH(CH₃)—C(CH₂CH₃)═NOCH₃ | EP-A 11 18 609 |
| I-11 | NHCH₃ | N | 2-Cl, 4-OCH(CH₃)—C(CH₃)═NOCH₂CH₃ | EP-A 11 18 609 |

TABLE II

| | |
|---|---|
| | (III) |
| 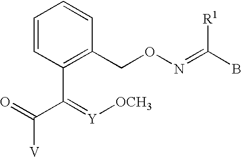 | |

| No. | V | Y | R¹ | B | Literature |
|---|---|---|---|---|---|
| II-1 | OCH₃ | CH | CH₃ | (3-CF₃)C₆H₄ | EP-A 370 629 |
| II-2 | OCH₃ | CH | CH₃ | (3,5-Cl₂)C₆H₃ | EP-A 370 629 |
| II-3 | NHCH₃ | N | CH₃ | (3-CF₃)C₆H₄ | WO-A 92/13830 |
| II-4 | NHCH₃ | N | CH₃ | (3-OCF₃)C₆H₄ | WO-A 92/13830 |
| II-5 | OCH₃ | N | CH₃ | (3-OCF₃)C₆H₄ | EP-A 460 575 |
| II-6 | OCH₃ | N | CH₃ | (3-CF₃)C₆H₄ | EP-A 460 575 |
| II-7 | OCH₃ | N | CH₃ | (3,4-Cl₂)C₆H₃ | EP-A 460 575 |
| II-8 | OCH₃ | N | CH₃ | (3,5-Cl₂)C₆H₃ | EP-A 463 488 |

TABLE III

| | |
|---|---|
| | (IV) |
| 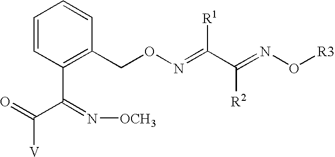 | |

| No. | V | R¹ | R² | R³ | Literature |
|---|---|---|---|---|---|
| III-1 | OCH₃ | CH₃ | CH₃ | CH₃ | WO-A 95/18789 |
| III-2 | OCH₃ | CH₃ | CH(CH₃)₂ | CH₃ | WO-A 95/18789 |
| III-3 | OCH₃ | CH₃ | CH₂CH₃ | CH₃ | WO-A 95/18789 |
| III-4 | NHCH₃ | CH₃ | CH₃ | CH₃ | WO-A 95/18789 |
| III-5 | NHCH₃ | CH₃ | 4-F—C₆H₄ | CH₃ | WO-A 95/18789 |
| III-6 | NHCH₃ | CH₃ | 4-Cl—C₆H₄ | CH₃ | WO-A 95/18789 |
| III-7 | NHCH₃ | CH₃ | 2,4-Cl₂—C₆H₃ | CH₃ | WO-A 95/18789 |
| III-8 | NHCH₃ | Cl | 4-F—C₆H₄ | CH₃ | WO-A 98/38857 |
| III-9 | NHCH₃ | Cl | 4-Cl—C₆H₄ | CH₂CH₃ | WO-A 198/38857 |
| III-10 | NHCH₃ | CH₃ | CH₂C(═CH₂)CH₃ | CH₃ | WO-A 97/05103 |
| III-11 | NHCH₃ | CH₃ | CH═C(CH₃)₂ | CH₃ | WO-A 97/05103 |
| III-12 | NHCH₃ | CH₃ | CH═C(CH₃)₂ | CH₂CH₃ | WO-A 97/05103 |
| III-13 | NHCH₃ | CH₃ | CH═C(CH₃)CH₂CH₃ | CH₃ | WO-A 97/05103 |
| III-14 | NHCH₃ | CH₃ | O—CH(CH₃)₂ | CH₃ | WO-A 97/06133 |
| III-15 | NHCH₃ | CH₃ | O—CH₂CH(CH₃)₂ | CH₃ | WO-A 97/06133 |
| III-16 | NHCH₃ | CH₃ | O(CH₃)═NOCH₃ | CH₃ | WO-A 917/15552 |
| III-17 | NHCH₃ | CH₃ | C₆H₅ | CH₃ | |

TABLE IV

| | |
|---|---|
| | (V) |
| 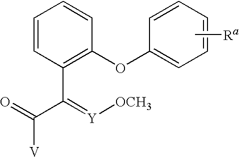 | |

| No. | V | Y | R^{a} | Literature |
|---|---|---|---|---|
| IV-1 | NHCH₃ | N | H | EP-A 398 692 |
| IV-2 | NHCH₃ | N | 3-CH₃ | EP-A 398 692 |
| IV-3 | NHCH₃ | N | 2-NO₂ | EP-A 398 692 |
| IV-4 | NHCH₃ | N | 4-NO₂ | EP-A 398 692 |
| IV-5 | NHCH₃ | N | 4-Cl | EP-A 398 692 |
| IV-6 | NHCH₃ | N | 4-Br | EP-A 398 692 |

Further examples of tested strobilurin compounds are also:

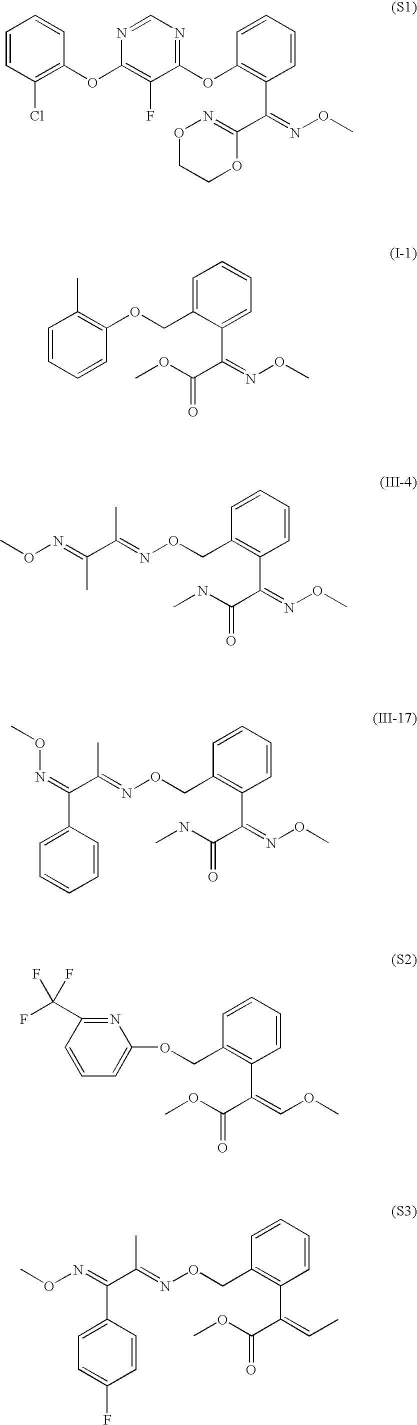

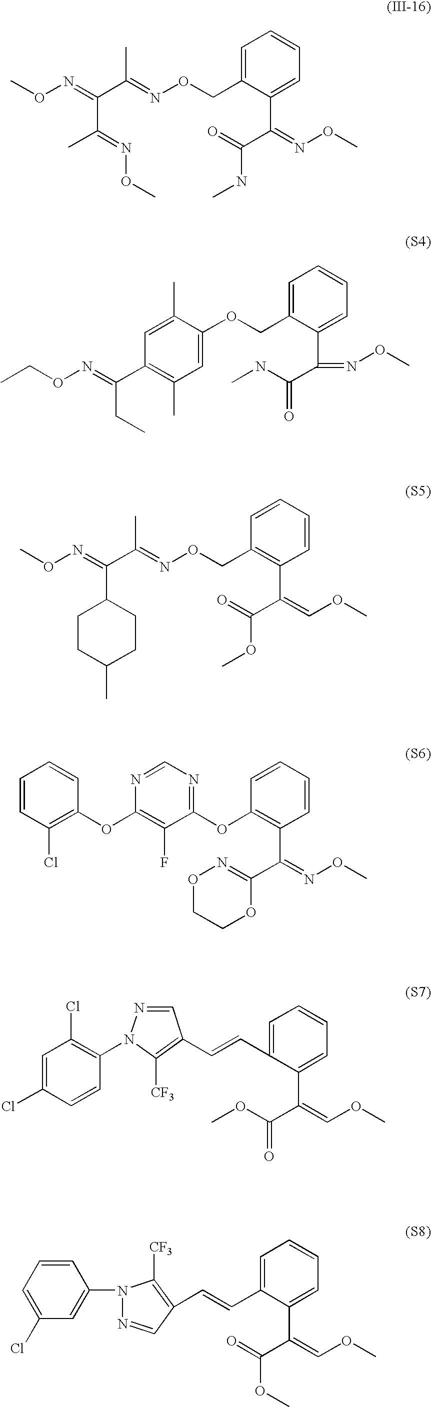

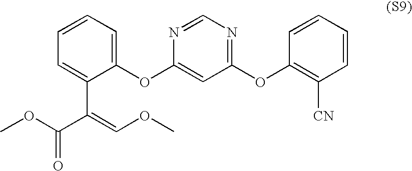

Strobilurin derivatives which may be mentioned for preferential use are furthermore:

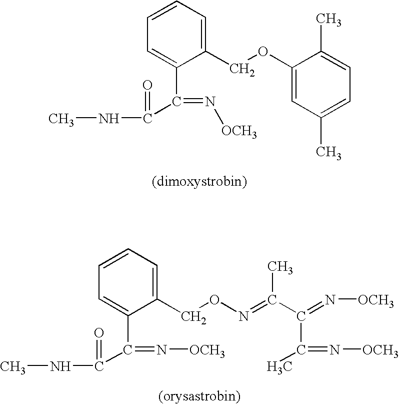

The invention moreover relates both to the use of a strobilurin derivative for the treatment of acute poisoning, and to the therapy and prophylaxis of chronically elevated iron levels in mammals, especially chronic disorders of humans.

Besides a strobilurin derivative, it is also possible to employ a further active ingredient (such as for example, ascorbic acid) for the treatment and/or prevention of disorders of iron metabolism.

The invention also relates very generally to a medicament comprising at least one strobilurin derivative, in particular a compound of the formula (I), and pharmaceutically suitable excipients.

A method for manufacturing a medicament comprising one or more strobilurins, in particular compounds of the formula (I), is likewise an aspect of the invention, where the compound of the formula (I) is mixed with a pharmaceutically suitable excipient, and this mixture is converted into a form suitable for administration.

The invention also relates to pharmaceutically acceptable salts of strobilurins. Pharmaceutically acceptable salts are frequently, because their solubility in water is higher than that of the basic compounds, particularly suitable for medical applications. These salts have a pharmaceutically acceptable anion or cation.

Suitable pharmaceutically acceptable acid addition salts of the compounds of the invention are, for example, salts of inorganic acids such as hydrochloric acid, hydrobromic, phosphoric, metaphosphoric, nitric and sulfuric acids, and of organic acids such as, for example, acetic acid, benzenesulfonic, benzoic, citric, ethanesulfonic, fumaric, gluconic, glycolic, lactic, lactobionic, maleic, malic, methanesulfonic, succinic, p-toluenesulfonic and tartaric acids.

Suitable pharmaceutically acceptable basic salts are in particular ammonium salts, alkali metal salts, especially sodium and potassium salts, and alkaline earth metal salts, especially magnesium and calcium salts, and salts of trometamol (2-amino-2-hydroxymethyl-1,3-propanediol), diethanolamine, lysine and ethylenediamine.

The term “physiologically functional derivative” used herein refers to any physiologically tolerated derivative of a compound of the invention, e.g. an ester, which, on administration to a mammal, such as, for example, mouse, rat or else human, is able to form, directly or indirectly, compounds of the formula (I) or an active metabolite thereof.

The physiologically functional derivatives to which the invention also relates also include so-called prodrugs of the compounds of the invention, as described for example by H. Okada et al. in Chem. Pharm. Bull. 1994, 42, 57-61. Such prodrugs can be metabolized in vivo to a compound of the invention. These prodrugs may themselves be active or not.

The compounds of the invention may frequently also exist in different polymorphous forms, e.g. as amorphous and crystalline polymorphous forms. The invention relates to all polymorphous forms of the compounds of the invention.

All references in the description of the present invention to “compounds according to the formulae (I) relate to compounds of the formulae (I) as described above, and their salts, solvates and physiologically functional derivatives. The same applies to compounds of the formulae (II), (III), (IV) and (V).

Various preparations can be provided for therapeutic use of the strobilurins. The amount of the compound of the invention necessary to achieve the desired biological effect normally depends on a plurality of factors, e.g. the chosen strobilurin compound, the mammal to be treated (e.g. mouse or human), the intended use (e.g. type of poisoning), the mode of administration and the age, sex and clinical condition of the patient.

The daily dose for humans is generally in the range from 1 mg to 100 mg (preferably from 3 mg to 80 mg) per day per kilogram of body weight.

An intravenous dose may be for example in the range from 1 mg to 50 mg/kg, which can be administered for example also as infusion of from 0.05 mg to 5 mg per kilogram per minute. Suitable infusion solutions for these purposes may comprise for example from 0.01 mg to 10 mg per milliliter. Single doses may comprise for example from 1 mg to 1000 mg of the active ingredient. Thus, ampoules for injections may comprise for example from 10 mg to 1000 mg.

It is possible to employ for single-dose formulations which can be administered orally, such as, for example, tablets or capsules, for example from 1 to 2000 mg, typically from 50 to 1000 mg, of the active compound.

For the therapy and prophylaxis of the abovementioned disorders of the Fe level it is possible to use the strobilurins, especially the compounds according to formula (I), themselves as compound, but they are preferably present with an acceptable excipient in the form of a pharmaceutical composition or preparation.

The excipient must be acceptable, i.e. it must be compatible with the other ingredients of the composition and must not be harmful to the health of the patient. The excipient may be for example a solid and/or a liquid and is preferably formulated with the active ingredient compound as single dose, for example as tablet, which may comprise from 0.05% to 95% by weight of the active ingredient.

Further pharmaceutically active substances may likewise be present, including further strobilurins of the invention, in particular compounds of the formula (I).

The pharmaceutical compositions of the invention can be manufactured by known methods. For example, the active ingredient(s) and pharmacologically acceptable excipients are vigorously mixed and then converted into the desired form.

Particularly suitable pharmaceutical compositions of the invention are those suitable for oral and peroral (e.g. sublingual), and parenteral (e.g. subcutaneous, intramuscular or intravenous) administration, but also those preferred for rectal or topical administration.

The invention also relates to coated formulations and slow-release formulations. A preferred embodiment of the invention relates to formulations resistant to acid and gastric juice. Suitable coatings resistant to gastric juice comprise for example cellulose acetate phthalate, polyvinyl acetate phthalate, hydroxypropylmethylcellulose phthalate and/or anionic polymers of methacrylic acid and/or of methyl methacrylates.

Suitable pharmaceutical preparations for oral administration may be in the form of separate units, for example as capsules, suckable tablets or tablets, each of which comprise a particular amount of the compound according to the formula (I). They may also be in the form of powders or granules, of solution or suspension in an aqueous or nonaqueous liquid. A formulation as an oil-in-water or water-in-oil emulsion is also possible.

These compositions can be prepared by any suitable pharmaceutical method which comprises a step in which the active ingredient and the excipient, which may also consist of a plurality of constituents, are brought into contact.

The compositions are generally manufactured by uniform and homogeneous mixing of the active ingredient with the liquid and/or finely divided solid adjunct, after which the product is, if necessary, shaped and subsequently packed.

It is thus possible for example to manufacture a tablet by compressing or shaping a powder or granules of the active compound, if appropriate with one or more additional excipients. Compressed tablets can also be manufactured by tableting the compound in free-flowing form, such as, for example, a powder or granules, if appropriate mixed with a binder, lubricant, diluent and/or dispersing means in a suitable machine. Shaped tablets can also be manufactured by shaping the active compound which is in powder form and is moistened with a liquid diluent in a suitable machine.

Pharmaceutical compositions for peroral administration are, for example, suckable tablets which comprise a compound according to formula (I) with a flavoring, normally sucrose and gum arabic, and pastilles which comprise the compound in an inert base such as gelatin or glycerol or sucrose and gum arabic.

Suitable pharmaceutical compositions for parenteral administration are preferably sterile aqueous preparations of strobilurins, in particular compounds according to formula (I), which are preferably isotonic with the blood of the intended recipient. These preparations are preferably administered intravenously. The administration can also take place subcutaneously, intramuscularly or intradermally as injection. These preparations can preferably be manufactured by mixing the compound with water and rendering the resulting solution sterile and isotonic with the blood of the recipient.

Injectable compositions of the invention generally comprise from 0.1 to 5% by weight of the active ingredient.

Suitable pharmaceutical compositions for rectal administration are in the form for example of single-dose suppositories, the manufacture of which is known in principle to the skilled worker.

Suitable pharmaceutical compositions for topical use on the skin are in particular ointment, cream, lotion, paste, spray, aerosol or oil. Examples of excipients which can be used are petrolatum, lanolin, polyethylene glycols, alcohols and combinations of these substances.

The active ingredient is generally present in a concentration of from 0.1 to 15% by weight of the composition, in particular from 0.5 to 2%.

Transdermal administration is also possible in principle. Suitable pharmaceutical compositions for transdermal uses may be in the form of single patches which are suitable for long-term close contact with the patient's epidermis. Such patches comprise the active ingredient dissolved in an optionally buffered aqueous solution and/or dispersed in an adhesive or dispersed in a polymer which gradually releases the active ingredient. A suitable active ingredient concentration is, for example, from 1% to 35%, preferably about 3% to 15%.

It has been possible to show in animal experiments on mice and rats that the strobilurins, and especially the compounds of the formula (I), are distinguished by favorable effects on disorders of iron metabolism. They influence inter alia the excretion of iron in the bile and the stool and reduce the absorption of iron. They therefore reduce in particular the iron level in the body (and in the blood) and are suitable for the prevention and treatment of iron-surplus disorders.

The compounds of the invention can be administered alone or in combination with one or more further pharmacologically active substances, which likewise for example have beneficial effects on disorders of iron metabolism or disorders associated therewith.

Dimoxystrobin, according to IUPAC nomenclature (E)-2(methoximino)-N-methyl-2-[α-(2,5-xylyloxy)-o-tolyl]acetamide, and orysastrobin, according to IUPAC (2E)-2(methoximino)-2-[2-[(3E,5E,6E)-5-(methoximino)-4,6-dimethyl-2 , 8-dioxa-3,7-diazanona-3,6-dien-1-yl]phenyl]-N-methylacetamide, have proved to be particularly suitable.

The examples specified below illustrate the invention:

### EXAMPLE 1

### Treatment of Rats with an Elevated Iron Level

Two grams of the phenylacetic acid derivative orysastrobin were prepared by known methods and purified by conventional methods.

Six-week old male Wistar rats (Elévage Janvier, France) were employed for the experiments. These rats, which were housed under conventional conditions (20-24° C., 30-70% humidity, 12 hours illumination, standard food) were divided into five groups of equal size.

The first group was treated with no iron compound and no strobilurin derivative.

The second group was treated only with 200 mg of the phenylacetic acid derivative orysastrobin (as suspension by gavage).

The third group received a single intramuscular injection of 50 mg/kg of an iron(III) hydroxide-dextran complex (Myofer® 100), 1 ml of the injection solution comprising 320 mg of the complex (equivalent to 195 mg of Fe(OH)₃).

The fourth group received both an injection of 50 mg/kg of the iron complex and, after 6 hours, a dose of 200 mg/kg of the strobilurin derivative by gavage.

The fifth group received both an injection of 50 mg/kg of the iron complex and, after 24 hours, a dose of 200 mg/kg of the strobilurin derivative by gavage.

24 hours before the start of the test, a blood sample was taken from all the rats, in each case at 8 o'clock in the morning of the preceding day, and was analyzed. The iron complex and/or the strobilurin derivative was administered at 8 o'clock in the morning of the day of the experiment. Further blood samples were taken from all the rats at 2 o'clock in the afternoon of the day of the experiment and at 8 o'clock in the morning of the next day, and were analyzed.

The following results were obtained:

An average iron content of 47.8 μmol/l was found in the blood in the untreated control group.

Measurement of the iron level in the blood in the second group of rats showed 20.6 μmol/l 6 hours after administration of the strobilurin derivative, and a value of 26.3 μmol/l after 24 hours.

The iron content found in the blood in the third group, which had received iron intramuscularly, was 153.9 μmol/l after 6 hours and 147.8 μmol/l after 24 hours.

Measurement of the iron level in the blood in the fourth group, which had received both intramuscularly a toxic intake of iron and a dose of the strobilurin derivative orysastrobin, showed 83.1 μmol/l after 6 hours.

The iron level in the blood determined in the fifth group after 24 hours was 91.9 μmol/l.

The results clearly illustrate that intramuscular intake of iron salts leads to a drastic rise in the iron concentration in the blood. It is additionally evident that the strobilurin derivative orysastrobin is particularly suitable for substantially reducing the iron content in the blood of the test animals within only a short time.

### EXAMPLE 2

### Treatment of HFE Mice

In order to demonstrate the therapeutic effect of the abovementioned compounds when iron metabolism is deranged, the following experiments were carried out with a particular breed of mouse, the mice exhibiting a mutation in the HFE gene.

This genetic defect leads to HFE mice having an elevated iron level in the liver, and this can serve as model of hemochromatosis disorder in humans. The HFE mice employed in the experiments were 12 to 23 weeks old (obtained from University College London, Department of Biochemistry and Molecular Biology).

The housing conditions of the mice corresponded to typical standards as have also been described in Example 1. In the experiments, investigations were carried out both with male HFE mice and with female HFE mice. In each case one group of animals received over a period of 7 days 2000 ppm of the phenylacetic acid derivative orysastrobin each day. The product was supplied freely in the food. The comparison group received only the regular food.

The following measurement results were found:

The control group of male mice had a serum iron level averaging 40.06 μmol/l iron after one week.

The serum iron level measured in the group of male mice treated with orysastrobin supplied via the food averaged 30.62 μmol/l after one week.

An iron level averaging 51.2 μmol/l was found after one week for the group of female comparison mice.

The female mice treated with orysastrobin were found to have an average iron level of 36.66 μmol/l.

This shows that the serum iron concentration in male mice is reduced by about 24%, and in female mice by about 28%, compared with the control groups. This can be interpreted as indicating that the strobilurin derivative is suitable for the therapy of a (hereditary) hemochromatosis.

### EXAMPLE 3

### Treatment of Rats with Dimoxystrobin

10 g of the phenylacetic acid derivative dimoxystrobin were prepared by known methods and purified by conventional methods.

Both 3-week old and 6-week old male Wistar rats (see Example 1) were employed for the subsequent experiments. These rats were divided into 6 equally sized groups of 10 rats. The rats were housed under conventional conditions.

The first group consisted of 3-week old rats. They received only the regular food over the entire period of the experiment.

The second group likewise consisted of 3-week old rats. The animals received 500 ppm (milligrams of test substance per kilogram body weight) of dimoxystrobin each day. It was supplied via the food.

The third group consisted of 6-week old rats which received no active ingredient via the food.

The fourth group consisted of 6-week old rats which received 500 ppm dimoxystrobin via the food each day.

The fifth group consisted of 3-week old rats which received no active ingredient.

The sixth group consisted of 3-week old rats which received 250 ppm of the test substance dimoxystrobin each day.

The iron concentrations in the blood were determined both after 2 days and after 7 days.

The average iron content found in the first untreated control group was 92.6 μmol/l and, after 7 days, 95.3 mmol/l.

In the second group of young rats which had received 500 mg of the strobilurin derivative, the measured blood level of iron was 35.8 μmol/l after 2 days and 33.6 μmol/l after 7 days.

In the third experimental group (adult rats without addition of active ingredient), the measured blood level of iron after 2 days was 43.1 μmol/l and after 7 days was 44.0 μmol/l.

In the fourth group of experimental animals (rats treated each day with 500 ppm of dimoxystrobin), the average iron level measured in the blood was 34.2 μmol/l after 2 days and 36 μmol/l after 7 days.

In the fifth group (control group of young rats), an iron level of 96.7 μmol/l was found after 2 days, and of 96.1 μmol/l after 7 days.

In the sixth group of experimental animals (young rats treated with 250 ppm dimoxystrobin each day), the measured level of iron in the blood after 2 days was 56 μmol/l and after 7 days was 59.7 μmol/l of blood.

In the rats treated with dimoxystrobin there was additionally found to be a distinct thickening of the duodenum, but this occurred only in the adult animals.

Owing to the smaller body weight and the greater metabolic rate, a greater uptake of the test substances was found in younger test rats. Administration of the strobilurin derivative leads to a significant reduction in the iron level in the blood.

These experiments illustrate that strobilurin derivatives such as dimoxystrobin can be used for therapy of disorders with an elevated iron level.

### EXAMPLE 4

In order to demonstrate the therapeutic effect for further compounds, the following tests were carried out with 10-week old male mice (C57BL/6 J Rj; Centre d'Elevage R. Janvier, France).

The housing conditions for the mice complied with typical standards. Investigations were carried out with male mice in the tests. In each case one group of animals received the respective strobilurin derivative orally each day for a period of 7 days. The respective product was supplied regularly via the food. The comparison group received the regular food exclusively.

The strobilurin derivatives (S2), (S4), (S5), (I-3), (III-4) and (III-17) described above were employed in each case. Investigations of the iron level-lowering activity were carried out on the mice with a dosage of from 5 mg/kg to 1000 mg/kg.

There was found to be a reduction in the Fe content in the blood of from 25 to 70% compared with the control animals after treatment with the abovementioned strobilurin derivatives for 7 days.

We claim:

1-9. (canceled)

10. A method for the treatment and/or prevention of disorders of iron metabolism in mammals comprising administering strobilurin derivatives and/or their physiologically tolerated salts for said treatment and/or prevention.

11. The method of claim 10, wherein a strobilurin derivative of formula (I) or a pharmaceutically acceptable salt is employed, 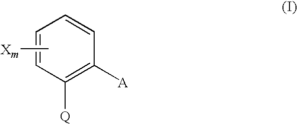
wherein
X is halogen, C₁-C₄-alkyl or trifluoromethyl;
m is 0 or 1;
Q is C(═CH—CH₃)—COOCH₃, C(═CH—OCH₃)—COOCH₃, C(═N—OCH₃)—CONHCH₃, C(═N—OCH₃)—COOCH₃, N(—OCH₃)—COOCH₃ or 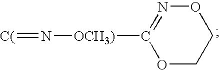
A is —O—B, —CH₂O—B, —CH₂S—B, —OCH₂—B, —CH≡CH—B, —CH₂O—N═C(R¹)—B, —CH₂O—N═C(R¹)—C(R²)═N—OR³, 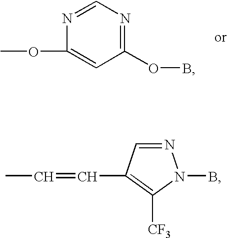
wherein
B is phenyl, naphthyl, 5-membered or 6-membered hetaryl or 5- or 6-membered heterocyclyl, comprising one to three N atoms and/or one O or S atom or one or two O and/or S atoms, wherein the ring systems are unsubstituted or substituted by one to three radicals R^{a}:
R^{a} is cyano, nitro, amino, aminocarbonyl, aminothiocarbonyl, halogen, C₁-C₆-alkyl, C₁-C₆-haloalkyl, C₁-C₆-alkylcarbonyl, C₁-C₆-alkylsulfonyl, C₁-C₆-alkylsulfinyl, C₃-C₆-cycloalkyl, C₁-C₆-alkoxy C₁-C₆-haloalkoxy, C₁-C₆-alkyloxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, phenyl, phenoxy, benzyl, benzyloxy, 5- or 6-membered heterocyclyl, 5- or 6-membered heterocyclyloxy, 5- or 6-membered hetaryl, 5- or 6-membered hetaryloxy, C(═NOR^{α})—OR^{β}, C(NOR^{α})—R^{β}, or OC(R^{α})₂—C(R^{β})═NOR^{β}, wherein the cyclic radicals, in turn, are unsubstituted or substituted by one to three radicals R^{b}:
R^{b} is cyano, nitro, halogen, amino, aminocarbonyl, aminothiocarbonyl, C₁-C₆-alkyl, C₁-C₆-haloalkyl, C₁-C₆-alkylsulfonyl, C₁-C₆-alkylsulfinyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl, C₃-C₆-cycloalkenyl, phenyl, phenoxy, phenylthio, benzyl, benzyloxy, 5- or 6-membered hetaryl, 5- or 6-membered hetaryloxy or C(═NOR^{α})—OR^{β};
R^{α}, R^{β} are hydrogen or C₁-C₆-alkyl;
R¹ is hydrogen, cyano, C₁-C₄-alkyl, C₁-C₄-haloalkyl, C₃-C₆-cycloalkyl, or C₁-C₄ alkoxy;
R² is phenyl, phenylcarbonyl, phenylsulfonyl, 5- or 6-membered hetaryl, 5- or 6-membered hetarylcarbonyl or 5- or 6-membered hetarylsulfonyl, wherein the ring systems are unsubstituted or substituted by one to three radicals R^{a}, or C₁-C₁₀-alkyl, C₃-C₆-cycloalkyl, C₂-C₁₀-alkenyl C₂-C₁₀-alkynyl, C₁-C₁₀-alkylcarbonyl, C₂-C₁₀-alkenylcarbonyl, C₃-C₁₀-alkynylcarbonyl, C₁-C₁₀-alkylsulfonyl, or C(R^{α})═NOR^{β}, wherein the hydrocarbon radicals of these groups are unsubstituted or substituted by one to three radicals R^{c}:
R^{c} is cyano, nitro, amino, aminocarbonyl, aminothiocarbonyl, halogen, C₁-C₆-alkyl, C₁-C₆-haloalkyl, C₁-C₆-alkylsulfonyl, C₁-C₆-alkylsulfinyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl, C₃-C₆-cycloalkyloxy, 5- or 6-membered heterocyclyl, 5- or 6-membered heterocyclyloxy, benzyl, benzyloxy, phenyl, phenoxy, phenylthio, 5- or 6-membered hetaryl, or 5- or 6-membered hetaryloxy and hetarylthio, wherein the cyclic groups in turn may be partly or completely halogenated or may carry one to three radicals R^{a}; and
R³ is hydrogen, C₁-C₆-alkyl, C₂-C₆-alkenyl, or C₂-C₆-alkynyl, wherein the hydrocarbon radicals of these groups are unsubstituted or substituted by one to three radicals R^{c}.

12. The method of claim 10, wherein a strobilurin derivative of formula (I) or a pharmaceutically acceptable salt is employed, 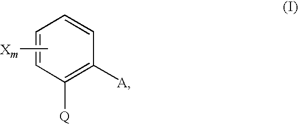
wherein
X is halogen, C₁-C₄-alkyl or trifluoromethyl;
m is 0 or 1;
Q is C(═CH—CH₃)—COOCH₃, C(═CH—OCH₃)—COOCH₃, C(═N—OCH₃)—CONHCH₃, C(═N—OCH₃)—COOCH₃ or N(—OCH₃)—COOCH₃;
A is —O—B, —CH₂O—B, —CH₂S—B, —OCH₂—B, —CH═CH—B, —CH₂O—N═C(R¹)—B or —CH₂O—N═C(R¹)—C(R²)═N—OR³, wherein
B is phenyl, naphthyl, 5-membered or 6-membered hetaryl or 5- or 6-membered heterocyclyl, comprising one to three N atoms and/or one O or S atom or one or two O and/or S atoms, wherein the ring systems are unsubstituted or substituted by one to three radicals R^{a}:
R^{a} is cyano, nitro, amino, aminocarbonyl, aminothiocarbonyl, halogen, C₁-C₆-alkyl, C₁-C₆-haloalkyl, C₁-C₆-alkylcarbonyl, C₁-C₆-alkylsulfonyl, C₁-C₆-alkylsulfinyl, C₃-C₆-cycloalkyl, C₁-C₆-alkoxy, C₁-C₆-haloalkoxy, C₁-C₆-alkyloxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, phenyl, phenoxy, benzyl, benzyloxy, 5- or 6-membered heterocyclyl, 5- or 6-membered heterocyclyloxy, 5- or 6-membered hetaryl, 5- or 6-membered hetaryloxy, C(═NOR^{α})—OR^{β} or OC(R^{α})₂—C(R^{β})═NOR^{β}, wherein the cyclic radicals in turn are unsubstituted or substituted by one to three radicals R^{b}:
R^{b} is cyano, nitro, halogen, amino, aminocarbonyl, aminothiocarbonyl, C₁-C₆-alkyl, C₁-C₆-haloalkyl, C₁-C₆-alkylsulfonyl, C₁-C₆-alkylsulfinyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl di-C₁-C₆-alkylaminothiocarbonyl, C₂-C₆-alkenyl, C₂-C₆-alkenyloxy, C₃-C₆-cycloalkyl, C₃-C₆-cycloalkenyl, phenyl, phenoxy, phenylthio, benzyl, benzyloxy, 5- or 6-membered hetaryl, 5- or 6-membered hetaryloxy or C(═NOR^{α})—OR^{β};
R^{α}, R^{β} are hydrogen or C₁-C₆-alkyl;
R¹ is hydrogen, cyano, C₁-C₄-alkyl, C₁-C₄-haloalkyl, C₃-C₆-cycloalkyl or C₁-C₄-alkoxy;
R² is phenyl, phenylcarbonyl, phenylsulfonyl, 5- or 6-membered hetaryl, 5- or 6-membered hetarylcarbonyl or 5- or 6-membered hetarylsulfonyl, wherein the ring systems are unsubstituted or substituted by one to three radicals R^{a}, or C₁-C₁₀-alkyl, C₃-C₆-cycloalkyl, C₂-C₁₀-alkenyl, C₂-C₁₀-alkynyl, C₁-C₁₀-alkylcarbonyl, C₂-C₁₀-alkenylcarbonyl, C₃-C₁₀-alkynylcarbonyl, C₁-C₁₀-alkylsulfonyl, or C(R^{α})═NOR^{β}, wherein the hydrocarbon radicals of these groups are unsubstituted or substituted by one to three radicals R^{c}:
R^{c} is cyano, nitro, amino, aminocarbonyl, aminothiocarbonyl, halogen, C₁-C₆-alkyl, C₁-C₆-haloalkyl, C₁-C₆-alkylsulfonyl, C₁-C₆-alkylsulfinyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl, C₃-C₆-cycloalkyloxy, 5- or 6-membered heterocyclyl, 5- or 6-membered heterocyclyloxy, benzyl, benzyloxy, phenyl, phenoxy, phenylthio, 5- or 6-membered hetaryl, or 5- or 6-membered hetaryloxy and hetarylthio, wherein the cyclic groups in turn may be partly or completely halogenated or may carry one to three radicals R^{a}; and
R³ is hydrogen, or
C₁-C₆-alkyl, C₂-C₆-alkenyl, or C₂-C₆-alkynyl, wherein the hydrocarbon radicals of these groups are unsubstituted or substituted by one to three radicals R^{c}.

13. The method of claim 11, wherein Q is C(═CH—OCH₃)—COOCH₃, C(═N—OCH₃)—COOCH₃, C(═N—OCH₃)—CO—NHCH₃ or N(—OCH₃)—COOCH₃.

14. The method of claim 11, wherein
m is 0;
A is —O—B, —CH₂O—B, —CH₂O—N═C(R¹)—B or CH₂—O—N═C(R¹)—C(R²═N—OR³);
B is phenyl, pyridyl, pyrimidinyl, pyrazolyl, or triazolyl, wherein these ring systems are substituted by one or two radicals R^{a};
R² is C₁-C₆-alkyl, C₂-C₁₀-alkenyl, or C₃-C₆-cycloalkyl, wherein these groups are unsubstituted or substituted by one or two radicals R^{b}';
R^{b′} is C₁-C₆-alkyl, C₃-C₆-cycloalkyl, C₁-C₆-alkoxy, C₁-C₆-haloalkoxy, benzyl, phenyl or phenoxy;
or phenyl which is unsubstituted or substituted by one or two radicals R^{a}; and
R³ is C₁-C₆-alkyl, C₂-C₁₀-alkenyl or C₂-C₁₀-alkynyl.

15. The method of claim 10, wherein an active ingredient of formula (II) is employed, 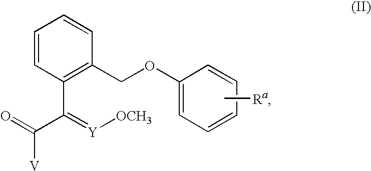
wherein V is OCH₃ or NH—CH₃, Y is N, and R^{a} is methyl, dimethyl or halogen.

16. The method of claim 10, wherein the disorder of iron metabolism comprises an acute poisoning.

17. The method of claim 10, wherein the disorder of iron metabolism comprises a chronic disorder.

18. The method of claim 10, comprising administering a further active ingredient in addition to the strobilurin derivative.

19. A pharmaceutical composition comprising a strobilurin derivative and/or a physiologically tolerated salt, and pharmaceutically suitable excipients and/or additives.

20. The composition of claim 19, which is in the form of an oral formulation.

21. The composition of claim 19, which is in the form of a parenteral formulation.

22. The composition of claim 19, comprising a strobilurin derivative of formula (I) and/or a physiologically tolerated salt, 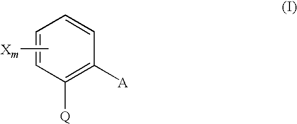
wherein
X is halogen, C₁-C₄-alkyl or trifluoromethyl;
m is 0 or 1;
Q is C(═CH—CH₃)—COOCH₃, C(═CH—OCH₃)—COOCH₃, C(═N—OCH₃)—CONHCH₃, C(═N—OCH₃)—COOCH₃, N(—OCH₃)—COOCH₃, or 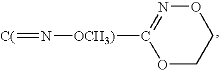
wherein
A is —O—B, —CH₂O—B, —CH₂S—B, —OCH₂—B, —CH═CH—B, —C≡C—B, —CH₂O—N═C(R¹)—B or —CH₂O—N═C(R¹)—C(R²)═N—OR³, 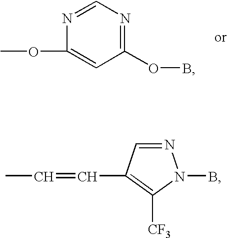
wherein
B is phenyl, naphthyl, 5-membered or 6-membered hetaryl or 5- or 6-membered heterocyclyl, comprising one to three N atoms and/or one O or S atom or one or two O and/or S atoms, wherein the ring systems are unsubstituted or substituted by one to three radicals R^{a}:
R^{a} is cyano, nitro, amino, aminocarbonyl, aminothiocarbonyl, halogen, C₁-C₆-alkyl, C₁-C₆-haloalkyl, C₁-C₆-alkylcarbonyl, C₁-C₆-alkylsulfonyl, C₁-C₆-alkylsulfinyl, C₃-C₆-cycloalkyl, C₁-C₆-alkoxy, C₁-C₆-haloalkoxy, C₁-C₆-alkyloxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, phenyl, phenoxy, benzyl, benzyloxy, 5- or 6-membered heterocyclyl, 5- or 6-membered heterocyclyloxy, 5- or 6-membered hetaryl, 5- or 6-membered hetaryloxy, C(═NOR^{α})—OR^{β}, C(═NOR^{α})—R^{β}, or OC(R^{α})₂—C(R^{β})═NOR^{β}, wherein the cyclic radicals in turn are unsubstituted or substituted by one to three radicals R^{b}:
R^{b} is cyano, nitro, halogen, amino, aminocarbonyl, aminothiocarbonyl, C₁-C₆-alkyl, C₁-C₆-haloalkyl, C₁-C₆-alkylsulfonyl, C₁-C₆-alkylsulfinyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl, C₃-C₆-cycloalkenyl, phenyl, phenoxy, phenylthio, benzyl, benzyloxy, 5- or 6-membered hetaryl, 5- or 6-membered hetaryloxy or C(═NOR^{α})—OR^{β};
R^{α}, R^{β} are hydrogen or C₁-C₆-alkyl;
R¹ is hydrogen, cyano, C₁-C₄-alkyl, C₁-C₄-haloalkyl, C₃-C₆-cycloalkyl, or C₁-C₄ alkoxy;
R² is phenyl, phenylcarbonyl, phenylsulfonyl, 5- or 6-membered hetaryl, 5- or 6-membered hetarylcarbonyl or 5- or 6-membered hetarylsulfonyl, wherein the ring systems are unsubstituted or substituted by one to three radicals R^{a}, C₁-C₁₀-alkyl, C₃-C₆-cycloalkyl, C₂-C₁₀-alkenyl, C₂-C₁₀-alkynyl, C₁-C₁₀-alkylcarbonyl, C₂-C₁₀-alkenylcarbonyl, C₃-C₁₀-alkynylcarbonyl, C₁-C₁₀-alkylsulfonyl, or C(R^{α})═NOR^{β}, wherein the hydrocarbon radicals of these groups are unsubstituted or substituted by one to three radicals R^{c}:
R^{c} is cyano, nitro, amino, aminocarbonyl, aminothiocarbonyl, halogen, C₁-C₆-alkyl, C₁-C₆-haloalkyl, C₁-C₆-alkylsulfonyl, C₁-C₆-alkylsulfinyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl, C₃-C₆-cycloalkyloxy, 5- or 6-membered heterocyclyl, 5- or 6-membered heterocyclyloxy, benzyl, benzyloxy, phenyl, phenoxy, phenylthio, 5- or 6-membered hetaryl, or 5- or 6-membered hetaryloxy and hetarylthio, wherein the cyclic groups in turn may be partly or completely halogenated or may carry one to three radicals R^{a}; and
R³ is hydrogen, or
C₁-C₆-alkyl, C₂-C₆-alkenyl, or C₂-C₆-alkynyl, wherein the hydrocarbon radicals of these groups are unsubstituted or substituted by one to three radicals R^{c}.

23. The composition of claim 22, comprising a strobilurin derivative of the formula (I) and/or a physiologically acceptable salt, wherein
X is halogen, C₁-C₄-alkyl or trifluoromethyl;
m is 0 or 1;
Q is C(═CH—CH₃)—COOCH₃, C(═CH—OCH₃)—COOCH₃, C(═N—OCH₃)—CONHCH₃, C(═N—OCH₃)—COOCH₃ or N(—OCH₃)—COOCH₃;
A is —O—B, —CH₂O—B, —CH₂S—B, —OCH₂—B, —CH═CH—B, —CH₂O—N═C(R¹)—B or —CH₂O—N═C(R¹)—C(R²)═N—OR³, wherein
B is phenyl, naphthyl, 5-membered or 6-membered hetaryl or 5- or 6-membered heterocyclyl, comprising one to three N atoms and/or one O or S atom or one or two O and/or S atoms, wherein the ring systems are unsubstituted or substituted by one to three radicals R^{a}:
R^{a} is cyano, nitro, amino, aminocarbonyl, aminothiocarbonyl, halogen, C₁-C₆-alkyl, C₁-C₆-haloalkyl, C₁-C₆-alkylcarbonyl, C₁-C₆-alkylsulfonyl, C₁-C₆-alkylsulfinyl, C₃-C₆-cycloalkyl, C₁-C₆-alkoxy, C₁-C₆-haloalkoxy, C₁-C₆-alkyloxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, phenyl, phenoxy, benzyl, benzyloxy, 5- or 6-membered heterocyclyl, 5- or 6-membered heterocyclyloxy, 5- or 6-membered hetaryl, 5- or 6-membered hetaryloxy, C(═NOR^{α})—OR^{β} or OC(R^{α})₂—C(R^{β})═NOR^{β}, wherein the cyclic radicals in turn are unsubstituted or substituted by one to three radicals R^{b}:
R^{b} is cyano, nitro, halogen, amino, aminocarbonyl, aminothiocarbonyl, C₁-C₆-alkyl, C₁-C₆-haloalkyl, C₁-C₆-alkylsulfonyl, C₁-C₆-alkylsulfinyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl di-C₁-C₆-alkylaminothiocarbonyl, C₂-C₆-alkenyl, C₂-C₆-alkenyloxy, C₃-C₆-cycloalkyl, C₃-C₆-cycloalkenyl, phenyl, phenoxy, phenylthio, benzyl, benzyloxy, 5- or 6-membered hetaryl, 5- or 6-membered hetaryloxy or C(═NOR^{α})—OR^{β};
R^{α}, R^{β} are hydrogen or C₁-C₆-alkyl;
R¹ is hydrogen, cyano, C₁-C₄-alkyl, C₁-C₄-haloalkyl, C₃-C₆-cycloalkyl, or C₁-C₄-alkoxy;
R² is phenyl, phenylcarbonyl, phenylsulfonyl, 5- or 6-membered hetaryl, 5- or 6-membered hetarylcarbonyl or 5- or 6-membered hetarylsulfonyl, wherein the ring systems are unsubstituted or substituted by one to three radicals R^{a}, or C₁-C₁₀-alkyl, C₃-C₆-cycloalkyl, C₂-C₁₀-alkenyl, C₂-C₁₀-alkynyl, C₁-C₁₀-alkylcarbonyl, C₂-C₁₀-alkenylcarbonyl, C₃-C₁₀-alkynylcarbonyl, C₁-C₁₀-alkylsulfonyl, or C(R^{α})═NOR^{β}, wherein the hydrocarbon radicals of these groups are unsubstituted or substituted by one to three radicals R^{c}:
R^{c} is cyano, nitro, amino, aminocarbonyl, aminothiocarbonyl, halogen, C₁-C₆-alkyl, C₁-C₆-haloalkyl, C₁-C₆-alkylsulfonyl, C₁-C₆-alkylsulfinyl, C₁-C₆-alkoxy, C₁-C₆-haloalkoxy, C₁-C₆-alkoxycarbonyl, C₁-C₆-alkylthio, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, C₃-C₆-cycloalkyl, C₃-C₆-cycloalkyloxy, 5- or 6-membered heterocyclyl, 5- or 6-membered heterocyclyloxy, benzyl, benzyloxy, phenyl, phenoxy, phenylthio, 5- or 6-membered hetaryl, or 5- or 6-membered hetaryloxy and hetarylthio, wherein the cyclic groups in turn may be partly or completely halogenated or may carry one to three radicals R^{a}; and
R³ is hydrogen, or
C₁-C₆-alkenyl, or C₂-C₆-alkynyl, wherein the hydrocarbon radicals of these groups are unsubstituted or substituted by one to three radicals R^{c}.

24. A method for manufacturing the composition of claim 19, comprising mixing the strobilurin derivative with pharmaceutically suitable excipients and/or additives, and converting this mixture into a form suitable for administration.
